(19) United States
(12) Patent Application Publication
(10) Pub. No.: US 2004/0242896 A1
(43) Pub. Date: Dec. 2, 2004

(54) Azole compounds as anti-fungal agents

(76) Inventors: Mohammad Salman, Gurgaon (IN); Rita Katoch, Chandigarh (IN); Ashwani Kumar Verma, New Delhi (IN); Jitendra Sattigeri, Gurgaon (IN); Ashok Rattan, New Delhi (IN)

Correspondence Address: RANBAXY INC., 600 COLLEGE ROAD EAST, SUITE 2100, PRINCETON, NJ, 08540, US

(21) Appl. No.: 10/483,906

(22) PCT Filed:
(86) PCT No.: PCT/IB02/02662

(30) Foreign Application Priority Data
Jul. 19, 2001 (IN) 778/DEL/2001
Dec. 21, 2001 (WO) PCT/IB01/02620

(51) Int. Cl. A61K 31/4196; C 07D 4 3/02

(52) U.S. Cl. 548/263.2; 514/383

(57) ABSTRACT

The present invention relates to the derivatives of specially substituted azole compounds which have improved anti-fungal activity as compared with presently available agents in this class and the processes for the preparation thereof. This invention also relates to pharmaceutical preparations containing the compounds of the present invention and their use in treating and/or preventing the fungal infections in mammals, preferably humans.

### FIELD OF THE INVENTION

The present invention relates to the derivatives of specially substituted azole compounds which have improved antifungal activity as compared with presently available agents in this class and the processes for the preparation thereof. This invention also relates to pharmaceutical preparations containing the compounds of the present invention and their use in treating and/or preventing the fungal infections in mammals, preferably humans.

### BACKGROUND OF THE INVENTION

Life threatening, systemic fungal infections continue to be a significant problem in health care today. In particular, patients who become “immunocompromised” as a result of diabetes, cancer, prolonged steroid therapy, organ transplantation anti-rejection therapy, the acquired immune deficiency syndrome (AIDS) or other physiologically or immunologically comprising syndromes, are especially susceptible to opportunistic fungal infections.

Since the 1950's and until recently, the key opportunistic fungal pathogens were Candida albicans, Asperigillus fumigatus, and Zygomycetes, which cause mucormycosis, a rapidly fatal infection especially in diabetic patients. Today, non-albicans Candida have become more frequent, as have other Aspergillus species. Candida species are now the fourth most common cause of nosocomial blood stream infection and they are associated with an extremely high mortality rate of 40%. From 1980 to 1990, the incidence of fungal infections in the US hospitals nearly doubled, from approximately 2.0 to 3.85% per 1000 patient days. The most marked increase in fungal infection rates occurred not only in transplant units or oncology centers, but also in surgical services. These changing patterns demonstrate that fungal infections are no longer limited to the most severly immunosuppressed patients.

During the past two decades, a substantial shift in the epidemiology of candidemia due to different Candida species has occurred. In the 1960's and 1970's, Candida albicans accounted for 85-90% of cases of candidemia. In 1999 however, only 42% of candidemia cases were caused by C. alibicans, while non-albicans candida accounted for the remainder.

Cryptococosis is a leading cause of morbidity among AIDS patients. The incidence of life threatening cryptococcal infection among these patients have been estimated to vary from 10 to 30%; 10-20% of these patients die during initial therapy and 30 to 60% patients succumb within a year. Penicillinium marneffei has been frequently isolated from HIV positive patents, especially in Southeast Asia.

The most common causative agent of mucormycosis is Rhizopus, a common bread mould that lives on any organic material. Other pathogens include Mucor, Rhizomucor and Absidia. Zygomycetes include twenty different fungi, all appearing the same histologically. The severely immunocompromised patient may become infected with Zygomycetes via respiratory inhalation.

Fusarium is the most prevalent plant fungus worldwide, and it is now recognized as human pathogen as well. Fusarium infections can occur in immunocompetent or immunosuppressed individuals. Fusarium infection is life-threatening and associated with a poor prognosis.

Penicillium marneffei is an environmental fungi that can cause serious, life-threatening infections in immunosuppressed patients. Penicillium marneffei has gained particular attention during the AIDS pandemic, as it may produce disease that is clinically indistinguishable from disseminated histoplasmosis.

Invasive aspergillosis has also become a leading cause of death, mainly among patients suffering from acute leukaemia or after allogenic bone marrow transfusion and after cytotoxic treatment of these conditions. It also occurs in patients with condition such as AIDS and chronic granulomatous disease. At present, only Amphotericin B and itraconazole are available for treatment of aspergillosis. In spite of their activity in-vitro, the effect of these drugs in-vivo against Aspergillus fumigatus remains low and as a consequence mortality from invasive aspergillosis remains high.

Over the last three decades important progress has been made in the therapy of systematic fungal infections. Although chemotherapeutic agents such as flucytosine and potassium iodide are effective against selected fungal diseases, the primary drugs used to treat systemic mycoses are amphotericin B and the azole compounds. Despite the general effectiveness of amphotericin B, it is associated with a number of complications and unique toxicities that limit its use. Furthermore, the drug is poorly absorbed from the gastrointestinal tract necessitating intravenous administration. In addition, amphotericin B penetrates poorly into cerebrospinal fluid (CSF) of both normal and inflamed meninges.

The problems associated with amphotericin B have stimulated search for new agents. Within the available drugs to treat fungal infections, the azole class appears to be most promising. This class of compounds inhibits the biosynthesis of ergosterol in fungi, which is the main constituent of fungal cell membrane. Of the various representative antifungals, early azoles used were clotrimazole, miconazole, and tioconazole, which were potent against a wide range of fungi pathogenic to human. Clortrimazole was the first oral azole proven to be effective in experimental and human mycosis. However, brief courses of treatment with clotrimazole lead to the induction of liver microsomal enzymes which in turn increase the metabolism of the drug, thereby diminishing its antifungal activity. In contrast, miconazole, which became available around the same time as clotrimazole, is not rapidly metabolized and is an effective intravenous therapy for many systemic fungal diseases. Unfortunately, the use of miconazole is limited by its multiple toxic effects.

The in-vitro activity of clotrimazole, miconazole and tioconazole was not well exhibited in in-vivo models due to poor oral bioavailability and metabolic vulnerability. Ketoconazole was the first drug that could be used against systemic fungal infection and successfully delivered through oral route. However, it was still quite susceptible to metabolic inactivation and also caused impotence and gynacomastia probably due to its activity against human cytochrome P450 enzymes.

Even with the advent of ketoconazole, the search for improved antifungal azole agents has continued due in part to concerns over the potential for toxicity and poor penetration into cerebrospinal fluid (CSF) associated with ketoconazole. Several azoles have been developed as topical agents primarily directed at superficial candidal and dermatophytic infections.

Fluconazole is the current drug of choice for treatment of severe infections caused by Candida species and C.neoformans. However, fluconazole has only weak activity against isolates of Aspergillus species [minimum inhibitory concentration (MIC) values of 400 μg/ml], since the drug has low potency (IC₅₀=4.8 μM) against lanosterol 14α-de-methylase, the target enzyme in the fungus. Itraconazole, another triazole antifungal compound, generally is more active than fluconazole in the treatment of aspergillosis, but its activity in the clinic remains mixed as it showed variable oral availability, low solubility and very high protein binding besides causing ovarian cancer in animals.

The development of the earlier compounds which included SCH 39304 (Genoconazole), SCH 42427 (Saperaconazole) and BAY R 8783 (Electrazole) had to be discontinued as a result of safety concerns. Another promising triazole, D0870, a derivative of fluconazole, exhibited significant variations in plasma pharmacokinetics besides having weak anti-Aspergillus activity. Other fluconazole derivatives in different stages of development include Voriconazole and ER 30346 (BMS 207147). Voriconazole also shows non-linear pharmacokinetics besides some concern regarding its ocular toxicity. ER 30346's anti-aspergillus activity, both in-vitro and in-vivo, is at best, only equal to itraconazole's activity. SCH 56592 is a hydroxylated analogue of itraconazole with potent in-vitro and in-vivo activity, but is undetectable in CSF even when the serum drug concentration after several days of treatment are 25 to 100 times above the MIC for the most resistant C. neoformans. Thus, the potent activity of SCH 56592 for C. neoformans is partially negated by its low concentration at the site of infection in the central nervous system. The above candidates of azoles are discussed in the following publications:

SCH 56592; Antimicrobial agents and chemotherapy, 40, 1910 (1996); 36^{th} Interscience Confernece on Antimicrobial agents and chemotherapy, September, 1996, New Orleans, Abst. To F-87-F-102.

TAK-187; 36^{th} Interscience Conference Antimicrobial agents and Chemotherapy, September, 1996, New Orleans, Abst. F 74; EP 567892.

TAK-456 and TAK-457; 40^{th} Interscience Conference on Antimicrobial agents and chemotherapy, Toronto, Canada, Abs. No. 1085 and 1086; U.S. Pat. No. 6,034,248.

ER-30346: Drugs of the Future, 21, 20 (1996).

Various derivatives of azole compounds have been covered in U.S. Pat. No. 5,371,101 assigned to Takeda. But none of them satisfies the medical needs completely, as they offer a limited spectrum of activity and low potency.

Thus, the antifungals available in the market suffer with drawbacks such as toxicity, narrow spectrum of activity and fungistatic profile rather fungicidal. Some of them also exhibit drug—drug interactions and, as a result, therapy becomes complex. In view of the high incidence of fungal infections in immunocompromised patients and the recent trends for the steady increase of the population of such patients, demands for new antifungal agents with broad spectrum of activity and good pharamcokinetic properties has increased. The continuing demand for safe and effective broad spectrum antifungal agent with favourable pharmacokinetic properties has spurred both the design and development of new systemically active antifungal triazoles.

Despite the therapeutic success of fluconazole and itraconazole, there remains a significant need for improved, broad spectrum, fungicidal rather than fungistatic, better tolerated, less toxic, safe at efficacious doses and more potent antifungal compounds with minimal potential for development of resistance among target fungi. Therefore, development of antifungal agents is still a big challenge.

### SUMMARY OF THE INVENTION

The present invention relates to new substituted azole compounds which can be utilized to treat and/or prevent the fungal infections in mammals, preferably in humans.

According to the present invention, there are provided compounds of Formula I, and its pharmaceutically acceptable salts, enantiomers, polymorphs, pharmaceutically acceptable solvates, diastereomers, N-oxides, prodrugs, or metabolites,

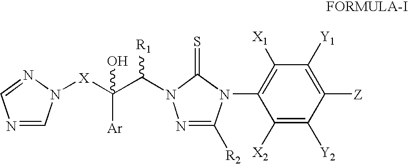

wherein X is selected from the group consisting of CH₂, CO, CS and SO₂;

Ar is a substituted phenyl group having one to three substituents independently selected from a halogen (e.g., fluorine, chlorine, bromine, or iodine), C₁-C₄ alkyl, halogenated lower (C₁-C₄) alkyl group and halogenated lower (C₁-C₄) alkoxy group such as 2,4-difluorophenyl, 2,4-dichlorophenyl, 4-chlorophenyl, 4-fluorophenyl, 2-chlorophenyl, 2-fluorophenyl, 4-trifluoromethylphenyl, 2-fluoro-4-chlorophenyl, 2-chloro-4-fluorophenyl, 4-trifluoromethoxyphenyl, 2,4,6-trifluorophenyl, 4-bromophenyl;

R₁ and R₂ are each independently selected from the group consisting of hydrogen, C₁-C₄ alkyl, C₁-C₄ alkoxy, amino, hydroxy, nitro, cyano, carboxyl, protected carboxyl, and SO₂R′ wherein R′ is hydrogen, alkyl or aryl;

X₁, X₂, Y₁, and Y₂ are independently selected from the group consisting of hydrogen, halogen, nitro, cyano, amino, sulphonyl, aryl, aralkoxy optionally substituted with one or more halogens (F, Cl, Br or I), C₁-C₄ alkyl, C₁-C₄ alkoxy, halogenated lower (C₁-C₄) alkyl group, halogenated lower (C₁-C₄) alkoxy group and carboxyl, or protected carboxyl and Z is aralkoxy optionally substituted with one or more halogens (F, Cl, Br, or I).

When R₁ is other than hydrogen, Formula I has two asymmetric centres and there are four possible enantiomers i.e. RR, RS, SR and SS. This invention relates to the mixture of enantiomers as well as individual isomers and the most preferred isomer in this situation is RR.

Pharmaceutically acceptable, non-toxic acid addition salts of the compounds of the present invention of Formula I, may be formed with inorganic or organic acids, by methods well known in the art.

It is also an object of the invention to provide a method for synthesis of the novel compounds.

It is further object of the present invention to provide compositions containing the novel compounds of the present invention in the treatment of fungal infections.

The present invention also includes within its scope prodrugs of the compounds of Formula I. In general, such prodrugs will be functional derivatives of these compounds which readily get converted in-vivo into defined compounds. Conventional procedures for the selection and preparation of suitable prodrugs are known.

The invention also includes pharmaceutically acceptable salts, polymorphs, pharmaceutically acceptable solvates, enantiomers, diastereomers, N-oxides, prodrugs, metabolites in combination with pharmaceutically acceptable carriers and optional excipients.

Other advantages of the invention will be set forth in the description which follows, and in part will be apparent from the description, or may be learned by the practice of the invention.

### DETAILED DESCRIPTION OF THE INVENTION

In order to achieve the above mentioned aspects and in accordance with the purpose of the invention as embodied and described herein, there is provided a process for the synthesis of compounds of Formula I, as shown in Scheme I.

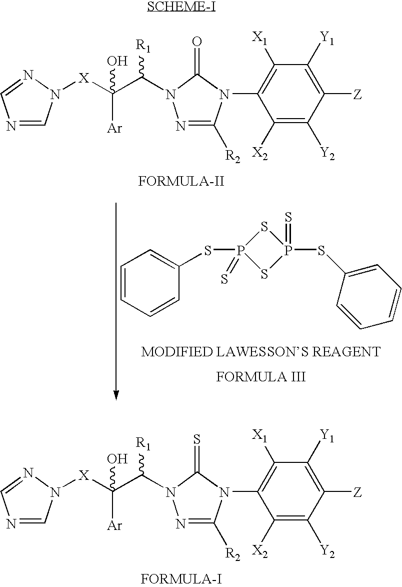

In Scheme I, there is provided a process for preparing a compound of Formula I, as shown above and its pharmaceutically acceptable salts, polymorphs, pharmaceutically acceptable solvates, enantiomers, diastereomers, N-oxides, prodrugs, or metabolites,

wherein X is selected from the group consisting of CH₂, CO, CS and SO₂;

Ar is a substituted phenyl group having one to three substituents independently selected from a halogen (e.g., fluorine, chlorine, bromine, or iodine) C₁-C₄ alkyl, halogenated lower (C₁-C₄) alkyl group and halogenated lower (C₁-C₄) alkoxy group such as 2,4-difluorophenyl, 2,4-dichlorophenyl, 4-chlorophenyl, 4-fluorophenyl, 2-chlorophenyl, 2-fluorophenyl, 4-trifluoromethylphenyl, 2-fluoro-4-chlorophenyl, 2-chloro-4-fluorophenyl, 4-trifluoromethoxyphenyl, 2,4,6-trifluorophenyl, 4-bromophenyl;

R₁ and R₂ are each independently selected from the group consisting of hydrogen, C₁-C₄ alkyl, C₁-C₄ alkoxy, amino, hydroxy, nitro, cyano, carboxyl, protected carboxyl, and SO₂R′ wherein R′ is hydrogen, alkyl or aryl;

X₁, X₂, Y₁, and Y₂ are independently selected from the group consisting of hydrogen, halogen, nitro, cyano, amino, sulphonyl, aryl, C₁-C₄ alkyl, C₁-C₄ alkoxy, halogenated lower (C₁-C₄) alkyl group, halogenated lower (C₁-C₄) alkoxy group and carboxyl, or protected carboxyl and Z is aralkoxy optionally substituted with one or more halogens (F, Cl, Br, or I);

which comprises reacting the appropriate oxo compound of Formula II, wherein X, Ar, R₁, R₂, Y, X₁, X₂, Y₁, Y₂ and Z have the same meanings as defined above, with modified Lawesson's reagent of Formula III, to afford the desired compound of Formula I. The oxo compound of Formula II may be prepared according to the procedure as disclosed in our published PCT application WO 01/66551 and is incorporated herein by reference. The modified Lawesson's reagent is prepared according to the procedure as disclosed by Masataka Yokohamna et al. in Synthesis, pp 827-829 (1984).

In the above scheme where specific solvent and specific modified Lawesson's reagent are mentioned, it is to be understood that other solvents and Lawesson's reagent or modification thereof may be used. Similarly, the reaction temperature and duration of the reaction may be adjusted according to the need. An illustrative list of some of the compounds according to the invention and capable of being produced by Scheme I include:

Compound No.1: 2-[(1R,2R)-2-(2,4-Difluorophenyl)-2-hydroxy-1-methyl-3-(1H-1,2,4-triazol-1-yl)propyl]4-[4-(2,4-dichlorobenzyloxy)phenyl]-3-(2H, 4H)-1,2,4-thiotirazolone;

Compound No.2: 2-[(1R,2R)-2-(2,4-Difluorophenyl)-2-hydroxy-1-methyl-3-(1H-1,2,4-triazol-1-yl)propyl]4-(4-benzyloxyphenyl)-3-(2H, 4H)-1,2,4-thiotriazolone;

The examples mentioned below demonstrate the general synthetic procedure as well as specific preparation for the preferred compound. The examples are given to illustrate the details of the invention and should not be constrained to limit the scope of the present invention.

The compounds were characterized using NMR, IR and were purified by chromatography. Crude products were subjected to column chromatographic purification using silica gel (100-200 or 60-120 mesh) as stationary phase.

### EXAMPLE 1

Typical Procedure for the Preparation of Compounds of Formula I

The oxo compound (1 mol) and Lawesson's Reagent (2 mol equivalent.) were dried under high vacuum for 10 min, flushed with nitrogen and heated to reflux in toluene for 15 hours. The reaction mixture was concentrated to dryness, re-dissolved in dichloromethane and purified by column chromatography (silica gel, 100-200 mesh), using dichloromethane-ethyl acetate mixtures (9.5:0.5 to 6:4) to afford the desired product in about 10% yield.

### EXAMPLE 2

Preparation of 2-[(1R,2R)-2-(2,4-Difluorophenyl)-2-hydroxy-1-methyl-3-(1H-1,2,4-triazol-1-yl)propyl]-4-[4-(2,4-dichlorobenzyloxy)phenyl]-3-(2H,4H)-1,2,4-thiotriazolone

A mixture of 2-[(1R,2R)-2-(2,4-Difluorophenyl)-2-hydroxy-1-methyl-3-(1H-1,2,4-triazol-1-yl)propyl]-4-[4-(2,4-dichlorobenzyloxy)phenyl]-3-(2H,4H)-1,2,4-triazolone (0.45 g) and Lawesson's Reagent (0.619 g, 2.0 m eq) was heated to reflux in toluene for about 15 hr.

The reaction mixture was concentrated under vacuum to give yellow semi-solid which was stirred with dichloromethane for about 10 min. The solid was filtered and washed with dichloromethane. The combined filtrate and washings were concentrated under vacuum to give a yellow semi-solid which was purified using column chromatography (using silica gel, 60-120 mesh followed by active alumina, basic) to give a white fluffy solid as the desired compound (0.095 g; 20.5%).

### EXAMPLE 3

Preparation of 2-[(1R,2R)-2-(2,4-Difluorophenyl)-2-hydroxy-1-methyl-3-(1H-1,2,4-triazol-1-yl)propyl]-4-(4-benzyloxyphenyl)-3-(2H,4H)-1,2,4-thiotriazolone

A mixture of 2-[(1R,2R)-2-(2,4-difluorophenyl)-2-hydroxy-1-methyl-3-(1H-1,2,4-triazol-1-yl)propyl]4-[4-benzyloxyphenyl]-3-(2H,4H)-1,2,4-triazolone (2.0 g) and Lawesson's Reagent (1.8 g, 1.2 m eq) was heated to reflux in toluene for about 15 hr.

The reaction mixture was concentrated under vacuum to give a yellow semi-solid which was stirred with dichloromethane for about 10 min. The solid was filtered and washed with dichloromethane. The combined filtrate and washings were concentrated under vacuum to give a yellow semi-solid which was purified using column chromatography (using silica gel, 60-120 mesh followed by active alumina, basic) to give a white fluffy solid as the desired compound (0.276 g; 13.38%).

Assignment of RR/SS was done on the basis of ¹HNMR analysis.

An illustrative list of some of the compounds of the invention which were synthesised by one or more of the above described methods is given below alongwith their ¹HNMR data. All ¹HNMR spectra were recorded on Brucker AMX 300 NMR machines (300 MHZ) using CDCl₃ as a solvent and TMS as an internal standard unless otherwise specified. All values are given in ppm.

Symbols in the examples have the meanings; s: singlet; d: doublet; t: triplet; q: quartet; dd: double doublet; m: multiplet; br: broad; J: coupling constant:

Compound No.1: 2-[(1R,2R)-2 (2,4-Difluorophenyl)-2-hydroxy-1-methyl-3-(1H-1,2,4-triazol-1-yl)propyl]-4-[4-(2,4-dichlorobenzyloxy)phenyl]-3-(2H, 4H)-1,2,4-thiotriazolone

m.p.: 130-132° C.

IR (KBr): 3431, 1616, 1512, 1245 and 831 cm⁻¹

NMR (CDCl₃): δ 1.35-1.33 (d, 3H, J=7.2 Hz, CH—CH₃), 4.393-4.346 (d, 1H, J=14 Hz, CH₂-Trz), 5.187-5.11 (s & d, 4H, OCH₂, OH, CH₂-Trz), 5.939 (q, 1H, J=6.6 Hz, CH—CH₃), 6.847 (t, 2H, J=10 Hz, ArH), 7.142-7.11 (d, 2H, J=8.7 Hz, ArH), 7.309 (m, 1H, ArH), 7.48 (m, 4H, ArH), 7.614 (m, 1H, J=8.7 Hz, ArH), 7.74 (s, 1H) and 7.94-7.92 (d, 2H, J=3.6 Hz)

Mass: m/z 603 (M⁺+1)

Compound No. 2: 2-[(1R,2R)-2 (2,4-Difluorophenyl)-2-hydroxy-1-methyl-3-(1H-1,2,4-triazol-1-yl)propyl]-4-(4-benzyloxyphenyl)-3-(2H, 4H)-1,2,4-thiotriazolone

m.p.: 155-157° C.

IR (KBr): 3429, 3121, 1615, 1511, 1246 and 964 cm⁻¹

NMR (CDCl₃): δ 1.343-1.32 (d, 3H, J=6.9 Hz, CH—CH₃), 4.376-4.329 (d, 1H, J=14 Hz, CH₂Trz), 5.15-5.10 (s & d, 3H, OCH₂Ph, CH₂Trz), 5.20 (s, 1H, br, D₂O exchangeable), 5.93 (q, 1H, J=6.9 Hz, CHCH₃), 6.84 (m, 2H, ArH), 7.135-7.106 (d,2H;J=8.9 Hz;Ar—H), 7.39 (m, 7H, ArH), 7.60 (q,1H, J=8.7 Hz, ArH) 7.73 (s, 1H) and 7.93-7.91 (d, 2H, 6.0 Hz)

Mass: m/z 535 (M⁺+1)

### Pharmacological Activity

Compounds of Formula I as shown herein, and their salts are useful in the curative or prophylactic treatment of fungal infections in animals, including humans. For example, they are useful in treating topical fungal infection in man caused by, among other organisms, species of Candida, Trichophyton, Microsporum or Epidermophyton in mucosal infections caused by C. albicans (eg. thrush and vaginal candidiasis). They can also be used in the treatment of systemic fungal infections caused by, for example, species of Candida (e.g. Candida albicans), Cryptococcus neoformans, Aspergillus fumigatus, Fusarium, Rhizopus or Penicillinium marneffei.

The compounds of the present invention have been found to have unexpectedly potent activity against clinically important filamentous species of fungi, besides increased spectrum. The compounds are fungicidal.

The in-vitro evaluation of the antifungal activity of the compounds (as shown in Table 1) can be performed by determining the minimum inhibitory concentration (MIC) which is the concentration of the test compound in Rosewell Park Memorial Institute (RPMI) 1640 liquid medium buffered with 3-(Morpholino)propanesulphonic acid (MOPS) to pH 7, at which there is significant inhibition of the particular fungi. In practice, the National Committee for Clinical Laboratory Standard (NCCLS) M27A document for Candida and Cryptococcus and M38P for Aspergillus was used to determine the MIC against yeast and filamentous fungi with suitable modifications for dermatophytes to other filamentous fungi. Three quality control strains were included each time the MIC were determined and readings recorded only when the Quality Control results fell into the acceptable range. After MIC results had been recorded, 100 μl from each of the well showing no growth was spread over Sabouraud Dextrose Aqar (SDA) to determine the minimum fungicidal concentration (MFC) as shown in Tables 2 and 3.

The in-vivo evaluation of the compound can be carried out at a series of dose levels by oral or I.V. injection to mice which are inoculated I.V. with the minimum lethal dose of Candida albicans, Cryptococcus neoformans or Aspergillus fumigatus by the tail vein. Activity is based on the survival of a treated group of mice after the death of an untreated group of mice. For Aspergillus and Cryptococcus infections, target organs were cultured after treatment to document the number of mice cured of the infection for further assessment of activity.

For human use, the antifungal compounds of the Formula I and their salts can be administered alone, but will generally be administered in admixture with a pharmaceutical carrier selected with regard to the intended route of administration and standard pharmaceutical practice. For example, they can be administered orally in the form of tablets containing such excipients as starch or lactose, or in capsules or ovules either alone or in admixture with excipients, or in the form of elixirs, solutions or suspensions containing flavouring or colouring agents. They can be injected parenterally, for example, intravenously, intramuscularly or subcutaneously. For parenteral administration, they are best used in the form of a sterile aqueous solution which may contain other substances, for example, enough salts or glucose to make the solution isotonic with blood.

The solubility of the compounds of the Formula I in an aqueous medium may be improved by complexation with a hydroxyalkyl derivative of a cyclodextrin in the preparation of an appropriate pharmaceutical composition.

For oral and parenteral administration to human patients, the daily dosage level of the antifungal compounds of the Formula I and their salts will be from 0.01 to 20 mg/kg (in single or divided doses) when administered by either the oral or parenteral routes. Thus tablets or capsules of the compound will contain from 5 mg to 0.5 gm of active compound for administration singly or two or more at a time, as appropriate. The physician in any event will determine the actual dosage which will be the most suitable for an individual patient and it will vary with age, weight and response of the particular patient. The above dosages are exemplary of the average case, there can, of course, be individual instances, where higher or lower dosage ranges are required and such are within the scope of this invention.

Alternatively, the antifungal compounds of the Formula I can be administered in the form of a suppository or pessary, or they may be applied topically in the form of a lotion, solution, cream, ointment or dusting powder. For example, they can be incorporated into a cream consisting of an aqueous emulsion of polyethylene glycols or liquid paraffin, or they can be incorporated, at a concentration between 1 and 10% into an ointment consisting of a white wax or white soft paraffin base together with such stabilizers and preservatives as may be required.

TABLE 1

MIC (μg/ml) of standard drugs and
compounds of the Invention

| | | | | Com- | Com- |
|---|---|---|---|---|---|
| | Fluco- | Ampho- | Itraco- | pound | pound |
| Organism | nazole | tericin B | nazole | No. 1 | No. 2 |
| Candida parapsilosis | 2 | 0.25 | 0.06 | <0.125 | 0.002 |
| 22019 (QC) | | | | | |
| Candida krusei 6258 | 32 | 0.25 | 0.5 | <0.125 | 0.125 |
| (QC) | | | | | |
| Paecilomyces variotti | 2 | 0.06 | 0.03 | <0.125 | 0.06 |
| 22319 QC) | | | | | |
| Candida albicans 1122 | 0.5 | 0.125 | 0.03 | <0.125 | — |
| Candida albicans | 8 | 0.125 | 0.125 | ≦0.125 | 0.03 |
| Y-01-19 | | | | | |
| Candida albicans1162 | >128 | 0.06 | 32 | ≧64 | — |
| Candida tropicalis 750 | 0.25 | 0.125 | 0.03 | <0.125 | 0.002 |
| Candida krusei 766.1 | 128 | 0.25 | 0.25 | 0.5 | 0.125 |
| Candida glabrata | 64 | 0.125 | 2 | 32 | 0.125 |
| 90030 | | | | | |
| Histoplasma | 2 | 0.25 | 0.25 | 0.5 | 0.03 |
| capsulatum | | | | | |
| Cryptococcus | 0.5 | 0.06 | 0.016 | <0.03 | 0.00025 |
| neoformans I | | | | | |
| Cryptococcus | 2 | 0.125 | 0.125 | <0.03 | 0.00025 |
| neoformans M 106 | | | | | |
| Aspergillus | >128 | 0.125 | 0.25 | <0.125 | 0.125 |
| fumigatus 1008 | | | | | |
| Aspergillus | >128 | 0.125 | 0.25 | <0.125 | 0.016 |
| fumigatus Si-I | | | | | |

While the present invention has been described in terms of its specific embodiments, certain modifications and equivalents will be apparent to those skilled in the art and are intended to be included within the scope of the present invention.

We claim:

1. A compound having the structure of Formula I, 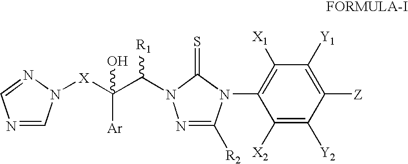
and its pharmaceutically acceptable salts, polymorphs, pharmaceutically acceptable solvates, enantiomers, diastereomers, N-oxides, prodrugs, or metabolites,
wherein X is selected from the group consisting of CH₂, CO, CS, and SO₂;
Ar is a substituted phenyl group having one to three substituents independently selected from a halogen (F, Cl, Br, or I), C₁-C₄ alkyl, halogenated lower (C₁-C₄) alkyl group and halogenated lower (C₁-C₄) alkoxy group;
R₁ and R₂ are each independently selected from the group consisting of hydrogen, C₁-C₄ alkyl, C₁-C₄ alkoxy, amino, hydroxy, nitro, cyano, carboxyl, protected carboxyl, and SO₂R′ wherein R′ is hydrogen, alkyl or aryl; and
X₁, X₂, Y₁, and Y₂ are independently selected from the group consisting of hydrogen, halogen, nitro, cyano, amino, sulphonyl, aryl, aralkoxy optionally substituted with one or more halogens (F, Cl, Br, or I), C₁-C₄, alkyl, C₁-C₄ alkoxy, halogenated lower (C₁-C₄) alkyl group, halogenated lower (C₁-C₄)alkoxy group and carboxyl, or protected carboxyl and Z is aralkoxy optionally substituted with one or more halogens (F, Cl, Br, or I).

2. A compound selected from the group consisting of:
2-[(1R,2R)-2 (2,4-Difluorophenyl)-2-hydroxy-1-methyl-3-(1H-1,2,4-triazol-1-yl)propyl]4-[4-(2,4-dichlorobenzyloxy)phenyl]-3-(2H,4H)-1,2,4-thiotriazolone (Compound No.1)
2-[(1R,2R)-2-(2,4-Difluorophenyl)-2-hydroxy-1-methyl-3-(1H-1,2,4-triazol-1-yl)propyl]4-(4-benzyloxyphenyl)-3-(2H,4H)-1,2,4-thiotriazolone (Compound No. 2)

3. A pharmaceutical composition comprising a pharmaceutically effective amount of a compound according to claims 1 or 2 or a physiologically acceptable acid additional salt thereof with a pharmaceutically acceptable carrier.

4. A method of treating or preventing fungal infection in a mammal comprising administering to said mammal a therapeutically effective amount of a compound having the structure of Formula I 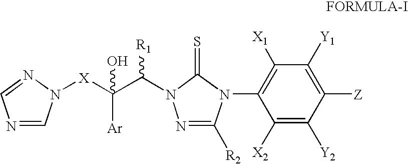
and its pharmaceutically acceptable salts, polymorphs, pharmaceutically acceptable solvates, enantiomers, diastereomers, N-oxides, prodrugs, or metabolites,
wherein X is selected from the group consisting of CH₂, CO, CS, and SO₂;
Ar is a substituted phenyl group having one to three substituents independently selected from a halogen (F, Cl, Br, or I), C₁-C₄ alkyl, halogenated lower (C₁-C₄) alkyl group and halogenated lower (C₁-C₄) alkoxy group;
R₁ and R₂ are each independently selected from the group consisting of hydrogen, C₁-C₄ alkyl, C₁-C₄ alkoxy, amino, hydroxy, nitro, cyano, carboxyl, protected carboxyl, and SO₂R′ wherein R′ is hydrogen, alkyl or aryl; and
X₁, X₂, Y₁, and Y₂ are independently selected from the group consisting of hydrogen halogen, nitro, cyano, amino, sulphonyl, aryl, aralkoxy optionally substituted with one or more halogens (F, Cl, Br or I), C₁-C₄, alkyl, C₁-C₄ alkoxy, halogenated lower (C₁-C₄) alkyl group, halogenated lower (C₁-C₄)alkoxy group and carboxyl, or protected carboxyl and Z is aralkoxy optionally substituted with one or more halogens (F, Cl, Br, or I).

5. A method of treating or preventing a fungal infection in a mammal comprising the step of administrating to said mammal a therapeutically effective amount of the pharmaceutical composition according to claim 3.

6. A process for preparing a compound having the structure of Formula I 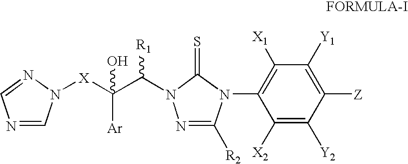
and its pharmaceutically acceptable salts, polymorphs, pharmaceutically acceptable solvates, enantiomers, diastereomers, N-oxides, prodrugs, or metabolites,
wherein X is selected from the group consisting of CH₂, CO, CS, and SO₂;
Ar is a substituted phenyl group having one to three substituents independently selected from a halogen (F, Cl, Br, or I), C₁-C₄ alkyl, halogenated lower (C₁-C₄) alkyl group and halogenated lower (C₁-C₄) alkoxy group;
R₁ and R₂ are each independently selected from the group consisting of hydrogen, C₁-C₄ alkyl, C₁-C₄ alkoxy, amino, hydroxy, nitrocyano, carboxyl, protected carboxyl, and SO₂R′ wherein R′ is hydrogen, alkyl or aryl; and
X₁, X₂, Y₁, and Y₂ are independently selected from the group consisting of hydrogen halogen, nitro, cyano, amino, sulphonyl, aryl, aralkoxy group optionally substituted with one or more halogens (F, Cl, Br, I), C₁-C₄, alkyl, C₁-C₄ alkoxy, halogenated lower (C₁-C₄) alkyl group, halogenated lower (C₁-C₄)alkoxy group and carboxyl, or protected carboxyl and Z is aralkoxy optimally substituted withone or more halogens (F, Cl, Br, or I),
which comprises reacting the oxo compound of Formula II (Scheme I) 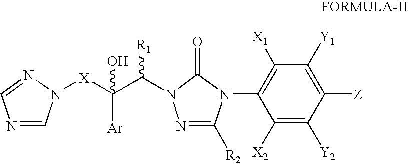
wherein X, Ar, R₁, R₂, X₁, X₂, Y₁, Y₂ and Z are the same as defined above, with modified Lawesson's reagent of Formula III 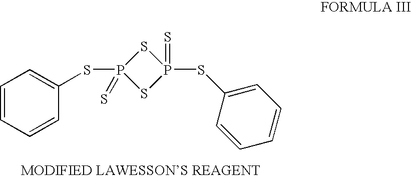
to afford the desired compound of Formula I.

7. A process for preparing a compound namely, 2-[(1R,2R)-2-(2,4-difluorophenyl)-2-hydroxy-1-methyl-3-(1H-1,2,4-triazol-1-yl)propyl]4-[4-(2,4-dichlorobenzyloxy)phenyl]-3-(2H,4H)-1,2,4-thiotriazolone and its pharmaceutically acceptable salts, polymorphs, pharmaceutically acceptable solvates, enantiomers, diastereomers, N-oxides, prodrugs, or metabolites, which comprises reacting the oxo-compound namely, 2-[(1R,2R)-2-(2,4-difluorophenyl)-2-hydroxy-1-methyl-3-(1H-1,2,4-triazol-1-yl)propyl]-4-[4-(2,4-dichlorobenzyloxy)phenyl]-3-(2H,4H)-1,2,4-triazolone with Lawesson's reagent.

8. A process for preparing a compound namely, 2-[(1R,2R)-2-(2,4-difluorophenyl)-2-hydroxy-1-methyl-3-(1H-1,2,4-triazol-1-yl)propyl]-4-(4-benzyloxyphenyl)-3-(2H,4H)-1,2,4-thiotriazolone and its pharmaceutically acceptable salts, polymorphs, pharmaceutically acceptable solvates, enantiomers, diastereomers, N-Oxides, prodrugs, or metabolites, which comprises reacting the oxo compound namely, 2-[(1R,2R)-2-(2,4-difluorophenyl)-2-hydroxy-1-methyl-3-(1H-1,2,4-triazol-1-yl)propyl]-4-[4-benzyloxyphenyl]-3-(2H,4H)-1,2,4-triazolone with Lawesson's reagent.
